(19) United States
(12) Plant Patent Application Publication
(10) Pub. No.: US 2002/0144326 P1
(43) Pub. Date: Oct. 3, 2002

(54) VERBENA PLANT NAMED 'DULCENA'

(76) Inventor: Henricus G.W. Stemkens, Hoorn (NL)

Correspondence Address: Larry W. Stults, Syngenta, Patent Department, P.O. Box 12257, Research Triangle Park, NC, 27709-2257, US

(21) Appl. No.: 09/819,241

(22) Filed: Mar. 28, 2001

(51) Int. Cl. A01H 5/00

(52) U.S. Cl. PLT/308

(57) ABSTRACT

A new Verbena plant particularly distinguished by its light blue flower, early flowering, and with a habit that is first semi-erect and later spreading.

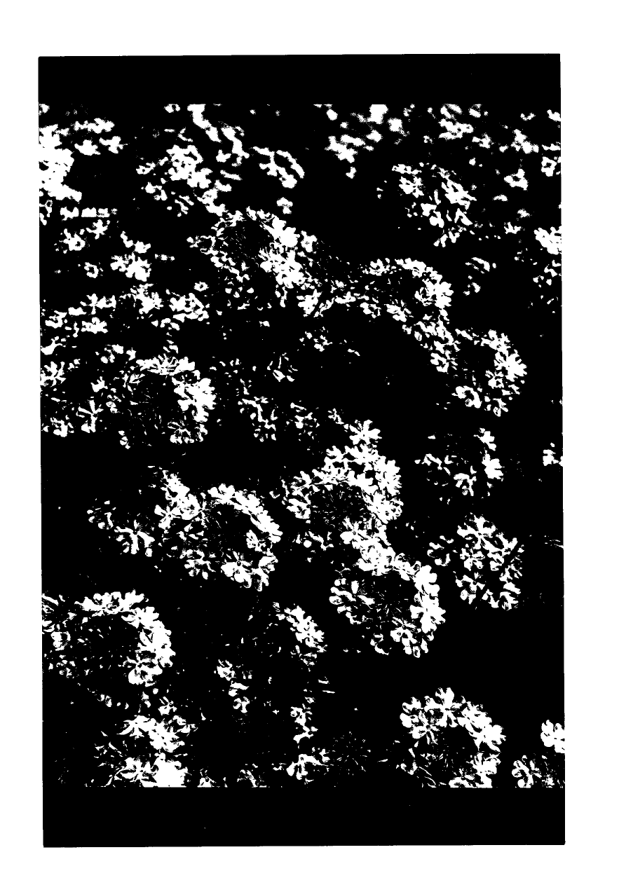

### BACKGROUND OF THE NEW PLANT

The present invention comprises a new distinct cultivar of Verbena, botanically known as Verbena*hybrida. The new cultivar is propagated from cuttings resulting from the cross of Y711 and U1012. Y711 is a blue flowering Verbena having a semi-erect habit. Y711 is not commercially available and is not known by any synonyms. U1012 a white flowering Verbena having a spreading habit. U1012 is not commercially available and is not known by any synonyms. Neither Y711 nor U1012 have been patented. As a result of this cross the present cultivar was created in 1997 in Enkhuizen, Netherlands and has been repeatedly asexually reproduced by cuttings in Enkhuizen, Netherlands and Sarrians, France over a three year period. It has been found to retain its distinctive characteristics through successive propagations, and this novelty appears to be firmly fixed. This new Verbena plant is an annual in most climatical zones in the US, only in zones 9 and 10 it is a perennial plant.

### DESCRIPTION OF THE DRAWING

This new Verbena plant is illustrated by the accompanying photographic drawing which shows blooms, buds and foliage of the plant in full colour, the colour shown being as true as can be reasonably obtained by conventional photographic procedures.

### DESCRIPTION OF THE NEW CULTIVAR

The following detailed descriptions set forth the distinctive characteristics of this new Verbena. The data which defines these characteristics were collected from asexual reproductions carried out in Enkhuizen, Netherlands. The plant history was taken on 14 weeks old plants, blossomed under natural light in a greenhouse. Colour readings were taken in the greenhouse under ambient light. Colour references are primarily to the RHS Colour Chart of The Royal Horticultural Society of London.

The plant:

Classification.—Botanical: Verbena*hybrida.

Parentage.—Female parent: A seedling named Y711 is one of our seedlings from our Y-generation of plants bred in 1995. Pollen parent A seedling named U1012 is one of our seedlings from our U-generation of plants bred in 1991.

Growth habit.—Semi-erect, later spreading.

Plant height.—18-28 cm.

Spreading area of plant.—40-70 cm.

Growth rate.—Hanging and vigorous.

Strength.—Very good.

Branching Character.—Very good branching.

Blooming period.—From April till November.

The stem:

Diameter.—2-2.5 mm.

Shape.—Tetragonal.

Anthocyan pigmentation.—Absent.

Length of internode.—20-40 mm, depending on the light where the plant is propagated.

Pubescence.—Some pubescence is present.

The foliage:

Phyllotaxis.—Opposite.

Shape of blade.—Broadly ovate, with deep incisions.

Texture.—Simple lobed.

Length.—25-45 mm.

Width.—20-40 mm.

Depth of incision.—10-20 mm.

Colour.—Upper side: Dark green 139A Lower side: Light green 143A.

Pubescence.—Some pubescence is present.

Length of leaf stem.—7-15 mm.

The bud:

Peduncle length.—30-50 mm, depending on season.

Size.—Diameter: 2 mm Length 8-10 mm.

Shape.—Long.

Colour.—Medium green 141A.

Sepals.—Colour Green 137D Form: Standing up.

The flower:

Direction.—Ascending.

Outward curvation of petal.—Medium curved.

Diamete.—20-26 mm.

Height.—16-18 mm.

Borne.—In a cluster.

Form.—Horizontal symmetric.

Cluster.—Umbel.

Colour.—Light Blue 85A.

Eye.—A very small (2 mm) green-white Eye is present on the base of all 5 petals.

No. of petals.—5.

Overlapping of petals.—Separate.

Shape of the petals.—Gamopetalous, grown together at the base of the petal. Each petal is heart shaped at the top.

Size of the petal.—Length: 7-9 mm Width 6-8 mm.

Spike.—Length: 20-30 mm Diameter 30-40 mm.

Calyx.—Length of 9-11 mm.

Anthocyan pigmentation of Calyx limb.—Absent.

No. of flowers per spike.—20-35.

Fragrance.—A very distinct rosy sweet fragrance is present.

Reproductive organs.—1 pistil, 5 stamens The plants are sterile, no seedset at all.

Roots:

Type of roots.—Fibrous, Roots start to grow on every part of the stem that contacts the soil, so not only at the nodes.

### Physiological and Ecological Characteristics

Good tolerance to heat and cold. Strong resistance to pests and diseases, particularly powdery mildew.

We claim:

1. A new and distinct variety of Verbena plant, substantially as herein illustrated and described, characterised particularly as to novelty by larger light blue flowers than other Verbena plants, which appear earlier on the plant in a larger number, and a growing habit that is more compact.
